(19) United States
(12) Patent Application Publication
(10) Pub. No.: US 2026/0151313 A1
(43) Pub. Date: Jun. 4, 2026

(54) METHODS AND MATERIALS TO IMPROVE THE SOLUBILITY OF AMIDE-BASED HYDROGEN PEROXIDE STABILIZERS

(71) Applicant: CAO Group, Inc., West Jordan, UT (US)

(72) Inventor: Steven D Jensen, South Jordan, UT (US)

(73) Assignee: CAO Group, Inc., West Jordan, UT (US)

(21) Appl. No.: 19/376,252

(22) Filed: Oct. 31, 2025

Related U.S. Application Data
(60) Provisional application No. 63/727,590, filed on Dec. 3, 2024.

(51) Int. Cl. A61K 8/42; A61Q 11/00

(52) U.S. Cl. CPC A61K 8/42; A61Q 11/00; A61K 2800/52

(57) ABSTRACT

A peroxide stabilizer for use in whitening compositions may comprise a primary amide stabilizer, such as hydroxyethyl urea, in solution with hydrogen peroxide. In comparison with carbamide, while having a lesser peroxide sequestration, hydroxyethyl urea is 2-3 times more soluble and therefore can provide a composition with greater stability while not sacrificing whitening power.

### CROSS-REFERENCES TO RELATED APPLICATIONS

The present invention claims the benefit of prior filed U.S. Provisional Application No. 63/727,590, filed on December 3, 2024 and incorporates the same by reference herein in its entirety.

### FIELD OF THE INVENTION

The present invention relates to the field of dentistry and more particularly relates to a stabilizer and method for stabilizing hydrogen peroxide.

### BACKGROUND OF THE INVENTION

It is well known in the art of chemistry and dentistry that hydrogen peroxide solutions are unstable and various means have been devised over the years to mitigate these stability issues to create usable dental whitening products. Such methods include and are not limited to keeping the pH in an acidic environment, minimizing the concentration of the solution, adding a secondary chemical stabilizer, and decreasing the storage temperature. Each of these strategies have been found to aid in increasing the stability of this valuable and useful oxidizer. For decades, the workhorse within the category of additive chemical stabilizers of hydrogen peroxide has been carbamide in the form of carbamide peroxide, especially in teeth whitening compositions. Carbamide, also known as urea (FIG. 1), forms an adduct with hydrogen peroxide and crystalizes out of solution at low temperature whereupon it can be rendered into a solid crystalline powder for storage and manufacture. Not only is it useful as an effective means of storage, but it also stabilizes the hydrogen peroxide and decreases the decomposition rate when manufactured into aqueous gel type products for teeth whitening.

Carbamide peroxide is only slightly soluble in water, hydrogen peroxide, and a host of solvents and humectants. It is well known to those in the art that carbamide peroxide tends to easily saturate compositions and therefore will often re-crystallize out

of solutions, whitening gels, whitening films and whitening strips. Simply put, there is a limit to how much carbamide peroxide you can place in any given composition. Whenever this limit is exceeded, the resultant composition will grow unwanted crystals, usually in the form of elongated prisms, especially when stored in inventory over prolonged periods of time. The growth of unwanted crystals in gelatinous whitening strips is especially detrimental because it tends to decrease the adhesive properties of the strip and, in worse case scenarios, the whitening strip will not stick to the teeth at all.

The low solubility and re-crystallization issues that are inherent with the use of carbamide peroxide are why some manufacturers try to avoid using carbamide peroxide. Despite all the excellent stability benefits carbamide provides, some manufacturers opt to use hydrogen peroxide directly in their whitening compositions and leave carbamide completely out of the formula. This practice creates additional problems as well, especially for teeth whitening strips. There are three inherent problems with using hydrogen peroxide alone:

a. Decreased Stability - teeth whitening compositions based solely on liquid hydrogen peroxide will decompose at a much faster rate than compositions stabilized with carbamide.

b. Dehydration - carbamide sequesters liquid hydrogen peroxide by forming a chemical adduct that significantly reduces the evaporation rate of liquid hydrogen peroxide in any whitening composition. Because hydrogen peroxide is a liquid similar to water, it will evaporate just like water until it eventually dries out enough that the strip becomes unusable. In order to compensate for this increased evaporation rate, some manufacturers add additional humectants such as propylene glycol and glycerin, which do not evaporate, so that the whitening strips are still pliable and can be applied to the teeth even after most of the liquid hydrogen peroxide has evaporated from the strip and they are no longer effective. This is done so that the whitening strip has sufficient adhesiveness to stick to the teeth regardless of whether it whitens or not.

c. Product Stability - the forced introduction of additional humectants causes the dried gel layer to never sufficiently solidify and therefore continue to slough and flow on the backing material. Some manufacturers that add non-evaporative

humectants to their whitening strip compositions incur additional problems to the storage longevity and effectiveness of their products. In order to compensate for this tendency to flow or slough on and off the strip some manufacturers add a plastic mesh layer on top of the backing material in order to inhibit the detrimental sloughing problem. This is highly inefficient because it requires an additional mesh layer and an additional manufacturing step that is costly.

There is a need in the teeth whitening industry for whitening compositions to have the extra stability and that additionally sequesters hydrogen peroxide thereby reducing the evaporation rate. This would allow the formulator to devise compositions that require little to no humectants such that the hazards of sloughing or flowing off the backing material is no longer an issue. There is also a need for a substitute for the old “workhorse” Carbamide that is inherently more soluble in water and hydrogen peroxide with the corresponding advantage of never detrimentally re-crystallizing out of a solution, gel, film, or strip.

The present invention is comprised of amide based stabilizers that contain at least one hydroxyl group per molecule, especially for use in teeth whitening compositions. The present invention represents a departure from the prior art in that these hydroxy amide based stabilizers are more soluble in water and hydrogen peroxide than the prior art stabilizer carbamide (urea) and will therefore limit the need for the excess use of non-evaporative humectants or solvents. The present invention allows for higher peroxide concentrations and a more stable whitening product without resorting to more expensive chemical and physical additions.

### SUMMARY OF THE INVENTION

In view of the foregoing disadvantages inherent in the known types of stabilized peroxide compositions, an improved peroxide composition may provide a composition that is stabilized with a primary amide that meets the following objectives: the resultant composition has greater stability and effectiveness over carbamide stabilized compositions without resorting to additional humectants or physical support.

The more important features of the invention have thus been outlined in order that the more detailed description that follows may be better understood and in order that the present contribution to the art may better be appreciated. Additional features of the invention will be described hereinafter and will form the subject matter of the claims that follow.

Many objects of this invention will appear from the following description and appended claims, reference being made to the accompanying drawings forming a part of this specification wherein like reference characters designate corresponding parts in the several views.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also, it is to be understood that the phraseology and terminology employed herein are for description and should not be regarded as limiting.

As such, those skilled in the art will appreciate that the conception, upon which this disclosure is based, may readily be utilized as a basis for the designing of other structures, methods, and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

In order to describe the manner in which the above-recited and other advantages and features of the invention can be obtained, a more particular description of the invention briefly described above will be rendered by reference to specific example embodiments thereof which are illustrated in the appended drawings. Understanding that these drawings depict only typical embodiments of the invention and are therefore not to be considered as limiting of its scope, the invention will be described and explained with additional specificity and detail using the accompanying drawings.

FIG. 1 is a molecular diagram showing the chemical structure of carbamide, also known as urea (prior art).

FIG. 2 is a molecular diagram showing the chemical structure of a primary amide

FIG. 3 is a molecular diagram showing the chemical structure of (2-hydroxyethyl) urea.

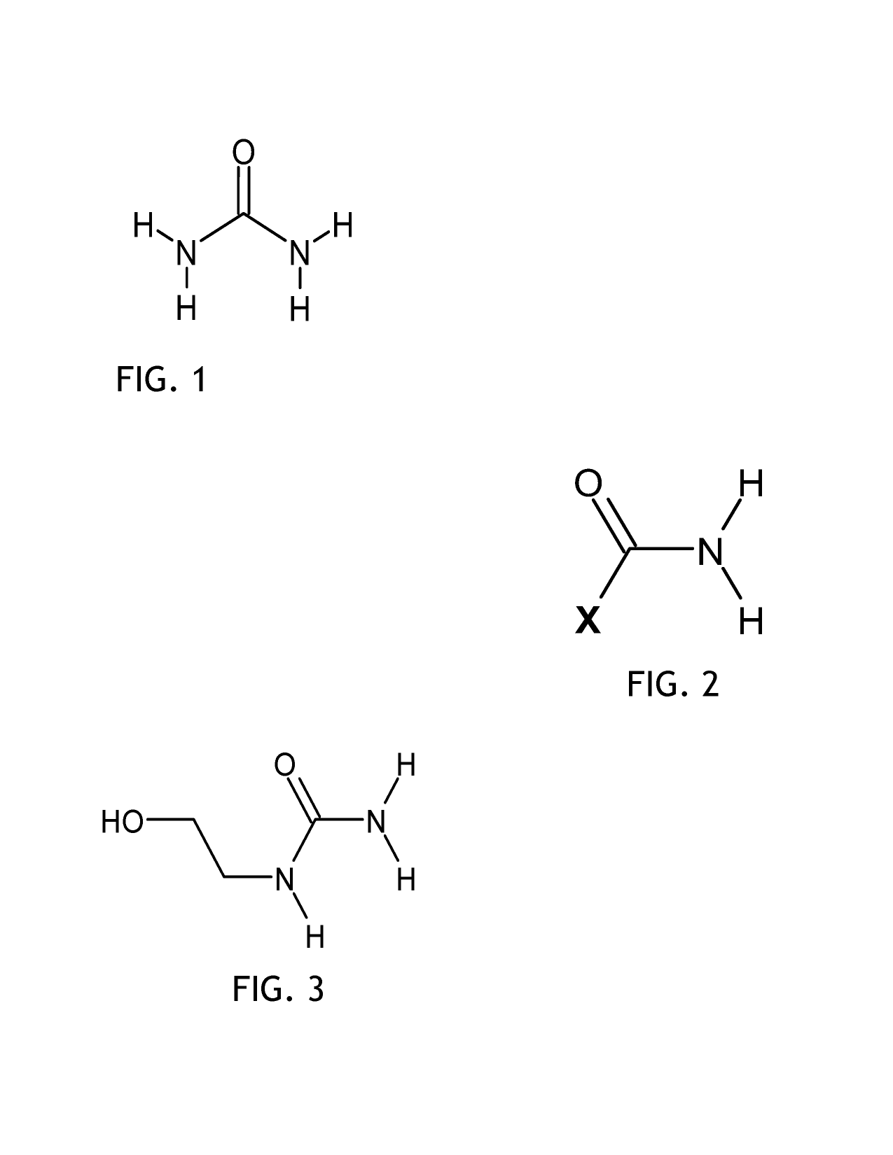

### DESCRIPTION

With reference now to the drawings, a preferred embodiment of the amide stabilizers for peroxide compositions is herein described. It should be noted that the articles “a”, “an”, and “the”, as used in this specification, include plural referents unless the content clearly dictates otherwise.

The most useful stabilizer and sequesterer of hydrogen peroxide is a primary amide, generically shown in FIG. 2. Substituting additional groups on the nitrogen decreases the efficacy; therefore, a secondary amide is less effective than a primary amide and a tertiary amide has almost no effectiveness at all. The preferred amide of the present invention is a primary amide where at least one or more hydroxyl groups are attached to the molecule. A secondary amide is not as preferred though it is within the scope of this patent.

The addition of a hydroxyl group greatly increase the solubility of the primary amide in both hydrogen peroxide and water, and in many cases the primary amide becomes soluble. This is a huge improvement over carbamide, which is only slightly soluble in water and hydrogen peroxide. In fact, loadings of up to 3 times greater in an aqueous solution of a hydroxyl primary amide results in no re-crystallization even when refrigerated overnight as compared to carbamide that cannot even dissolve 1/3 the amount before becoming saturated.

FIG. 3 is a drawing of the chemical structure (2-hydroxyethyl) urea. (2-hydroxyethyl) urea peroxide is an excellent candidate to demonstrate the advantages of the present invention over the prior art carbamide peroxide.

Carbamide (urea) has two primary amides per molecule and it was previously believed necessary to have at least 2 amide groups per molecule in order to sequester one molecule of hydrogen peroxide in a 1:1 molar ratio, but this was later disproven by experiments with hydroxyethyl urea. It was found that a single primary amide could sequester one or more hydrogen peroxide molecules. This means by the same token of understanding that carbamide is capable of sequestering 2 or more hydrogen peroxide molecules, more than originally anticipated. This was demonstrated when measuring the available peroxide of a gelatinous teeth whitening strip while it was dried into a film on a parafilm backing layer. When comparing carbamide (urea) versus hydroxyethyl urea at the same concentration in 2 separate gels dried into a film. The results showed that carbamide could sequester and stabilize more hydrogen peroxide than hydroxyethyl urea by about 25%. While this is no surprise because carbamide has two primary amides per molecule as compared to one primary amide and one secondary amide per molecule for hydroxyethyl urea. Therefore, what is observed is an equilibrium of peroxide sequestering with respect to the number of primary and secondary amides per molecule when loaded at equal concentrations within a teeth whitening composition. It also demonstrates that secondary amides are significantly less efficient at sequestering hydrogen peroxide than primary amides. Hydrogen peroxide stabilizers that contain only secondary amides bare similar inefficiencies. For this reason, the primary amide is the preferred stabilizer for hydrogen peroxide.

While carbamide (urea) is more efficient at sequestering hydrogen peroxide than hydroxyethyl urea, the superiority of hydroxyethyl urea is demonstrated by the fact 2-3 times more hydroxyethyl urea may be dissolved in water and hydrogen peroxide than carbamide (urea); and this without the risk of a manufactured teeth whitening strip detrimentally re-crystallizing during long and short term storage. The advantage will always favor the increase in the solubility of the peroxide stabilizer, because one can simply load more hydroxy amide stabilizer into a composition than you can with a prior art amide stabilizer that has limited solubility. Another benefit of having a higher concentration of hydroxyethyl urea in a teeth whitening strip is it helps solidify the gelatinous whitening layer so that it minimizes the problems of sloughing or flowing off the polymeric backing material. Additionally, significant amounts of non-evaporative solvents and humectants do not have to be added to the composition so the resultant strips do not prematurely dry out before use. We have found with hydroxyethyl urea peroxide compositions comprised of packaged gelatinous teeth whitening strips that during storage at least 70% of the compositional peroxide active ingredient is still available for teeth whitening after 1 month of cold storage at 4 Celsius.

As can be observed by the hydroxyethyl urea peroxide example, simply by adding one or more hydroxyl groups per molecule to a primary amide increases the solubility in water and hydrogen peroxide significantly, such that higher loadings of stabilizer can be achieved in teeth whitening compositions. Referring again to FIG. 2, a substituent represented by the letter X may be attached to a primary amide. The letter X represents substituents which may include a single chemical substituent, a group of substituents, or a plurality of groups of substituents, so long as the substituent contains at least one hydroxyl group. This is illustrated as an example, and as a non-exclusive preferred embodiment, in FIG. 3, hydroxyethyl urea. This in turn increases the overall whitening strip shelf life, decreases the peroxide decomposition rate, decreases the peroxide evaporation rate, minimizes the risk of sloughing and minimizes the risk of a re-crystallization event during short and long term storage.

Although the present invention has been described with reference to preferred embodiments, numerous modifications and variations can be made and still the result will come within the scope of the invention. The embodiments described are to be considered in all respects only as illustrative and not restrictive. No limitation with respect to the specific embodiments disclosed herein is intended or should be inferred. Therefore, the scope of the invention is indicated by the appended claims rather than by the foregoing description. All changes which come within the meaning and range of equivalency of the claims are to be embraced within their scope.

What is claimed is:

1. A primary amide stabilizer for hydrogen peroxide, the primary amide stabilizer comprising the chemical structure:
where X is a substituent selected from the set of substituents consisting of any single chemical substituent, a group of substituents and groups of substituents, the substituent comprising a chemical moiety that further comprises at least one hydroxyl groups.

2. The stabilizer of claim 1, the primary amide stabilizer for hydrogen peroxide being hydroxyethyl urea.

3. A teeth whitening composition comprising a primary amide stabilizer for the purpose of stabilizing a peroxide, the primary amide stabilizer comprising the chemical structure:
where X is a substituent selected from the set of substituents consisting of any single chemical substituent, a group of substituents and groups of substituents, the substituent comprising a chemical moiety that further comprises at least one hydroxyl groups.

4. The stabilizer of claim 3, the primary amide stabilizer for hydrogen peroxide being hydroxyethyl urea.
